(19) United States
(12) Patent Application Publication
(10) Pub. No.: US 2014/0074169 A1
(43) Pub. Date: Mar. 13, 2014

(54) SPINAL CORRECTION SYSTEM AND METHOD

(71) Applicants: Jacob Shorez, Marion, IA (US); Bradley E. Steele, St. Louis Park, MN (US); Matthew J. Van Nortwick, Portage, MI (US)

(72) Inventors: Jacob Shorez, Marion, IA (US); Bradley E. Steele, St. Louis Park, MN (US); Matthew J. Van Nortwick, Portage, MI (US)

(73) Assignee: WARSAW ORTHOPEDIC, INC., Warsaw, IN (US)

(21) Appl. No.: 13/614,581

(22) Filed: Sep. 13, 2012

(51) Int. Cl. A61B 17/70

(52) U.S. Cl. 606/264

(57) ABSTRACT

A spinal correction system comprises a fixation element including a proximal portion and a distal portion. The proximal portion includes a wall that defines a cavity and at least one post including a first portion and a second portion having a center. The fixation element further includes a locking element engageable with the wall to dispose the second portion in the cavity. A connector defines a first cavity configured for disposal of a longitudinal element and a second cavity configured for disposal of the first portion. The fixation element is moveable between a first orientation such that the first portion is rotatable to a selected angle about the center in a plurality of planes relative to the distal portion and a second orientation such that the locking element fixes the first portion in a selected position relative to the distal portion. Methods of use are disclosed.

### TECHNICAL FIELD

The present disclosure generally relates to medical devices for the treatment of musculoskeletal disorders, and more particularly to a surgical system and method for correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods and bone screws, for stabilization of a treated section of a spine. This disclosure describes an improvement over these prior art technologies.

### SUMMARY

Accordingly, a spinal correction system is provided. In one embodiment, the spinal correction system comprises at least one longitudinal element. At least one fixation element includes a proximal portion and a distal portion configured for penetrating tissue. The proximal portion includes a wall that defines a cavity and at least one post including a first portion and a second portion having a center. The at least one fixation element further includes a locking element engageable with the wall to dispose the second portion in the cavity. A connector defines a first cavity configured for disposal of the at least one longitudinal element and a second cavity configured for disposal of the first portion. The at least one fixation element is moveable between a first orientation such that the first portion is rotatable to a selected angle about the center in a plurality of planes relative to the distal portion and a second orientation such that the locking element fixes the first portion in a selected position relative to the distal portion.

In one embodiment, a method for treating a spinal disorder is provided. The method comprises the steps of: providing a spinal correction system comprising: a longitudinal element, at least one fixation element including a proximal portion and a distal portion, the proximal portion including a wall that defines a cavity and at least one post including a first portion and a second portion having a center, the at least one fixation element further including a locking element engageable with the wall to retain the second portion in the cavity, and a connector defining a first cavity and a second cavity; attaching the distal portion with tissue; provisionally engaging the locking element with the wall; disposing the longitudinal element in the first cavity; rotating the first portion in a first orientation to a selected angle about the center in a plurality of planes relative to the distal portion to a selected position such that the first portion is aligned with the second cavity; engaging the locking element with the wall to fix the first portion in a second orientation in the selected position; and disposing the first portion in the second cavity.

In one embodiment, the spinal correction system comprises at least one longitudinal element; at least one fixation element including a proximal portion and a distal portion configured for penetrating tissue, the proximal portion including a collar having a threaded outer surface and defining a concave socket, the proximal portion further including a post including a shaft having an inner threaded surface and being integrally connected with a ball having a center, the at least one fixation element further including a locking nut having an inner threaded surface that defines a circular opening having a radial perimeter defining a range of motion of the first portion, the threaded inner surface of the locking nut being configured to mate with the threaded outer surface of the collar to dispose the ball in the socket, the at least one fixation element further including a compression element disposed between the collar and the ball, the compression member including a first surface engageable with the collar and a second concave surface engageable with the ball, the second surface including a stepped surface; a connector defining a first cavity configured for disposal of the at least one longitudinal element and a second cavity configured for disposal of the first portion, the first cavity being disposed in a transverse orientation relative to the second cavity; and an elongated reduction element having a threaded portion engageable with the inner threaded surface of the first portion, wherein the fixation element is moveable between a first orientation such that the shaft is rotatable to a selected angle about the center in a plurality of planes relative to the distal portion and a second orientation such that the locking element fixes the shaft in a selected position relative to the distal portion.

### BRIEF DESCRIPTION OF THE DRAWINGS

The present disclosure will become more readily apparent from the specific description accompanied by the following drawings, in which:

FIG. 1 is a perspective view of a spinal correction system in accordance with the principles of the present disclosure;

FIG. 2 is a perspective view of the spinal correction system shown in FIG. 1 with parts separated;

FIG. 3 is a break away cross section view of components of the spinal correction system shown in FIG. 1;

FIG. 4 is a perspective view of a component of the spinal correction system shown in FIG. 1;

FIG. 5 is a perspective view of components of the spinal correction system shown in FIG. 1; and

FIG. 6 is a perspective view of the spinal correction system shown in FIG. 1 disposed with vertebrae.

Like reference numerals indicate similar parts throughout the figures.

### DETAILED DESCRIPTION

The exemplary embodiments of the spinal correction system and related methods of use are discussed in terms of medical devices for the treatment of musculoskeletal disorders and more particularly, in terms of a spinal implant system. It is envisioned that the spinal implant system may be employed in applications for correction of deformities, such as, for example, kyphosis and scoliosis. It is contemplated that one or all of the components of the surgical system may be disposable, peel-pack, pre-packed sterile devices. One or all of the components of the system may be reusable. It is envisioned that one or all of the components of the system may be modified in vivo. In one embodiment, the system provides adjustment and/or alignment of its component parts through multiple degrees of freedom and has locking capability to lock one or all of the components of the system in a selected position. The system may be configured as a kit with multiple sized and configured components to allow for adjustment of, for example, the height of the system relative to vertebra and/or components.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

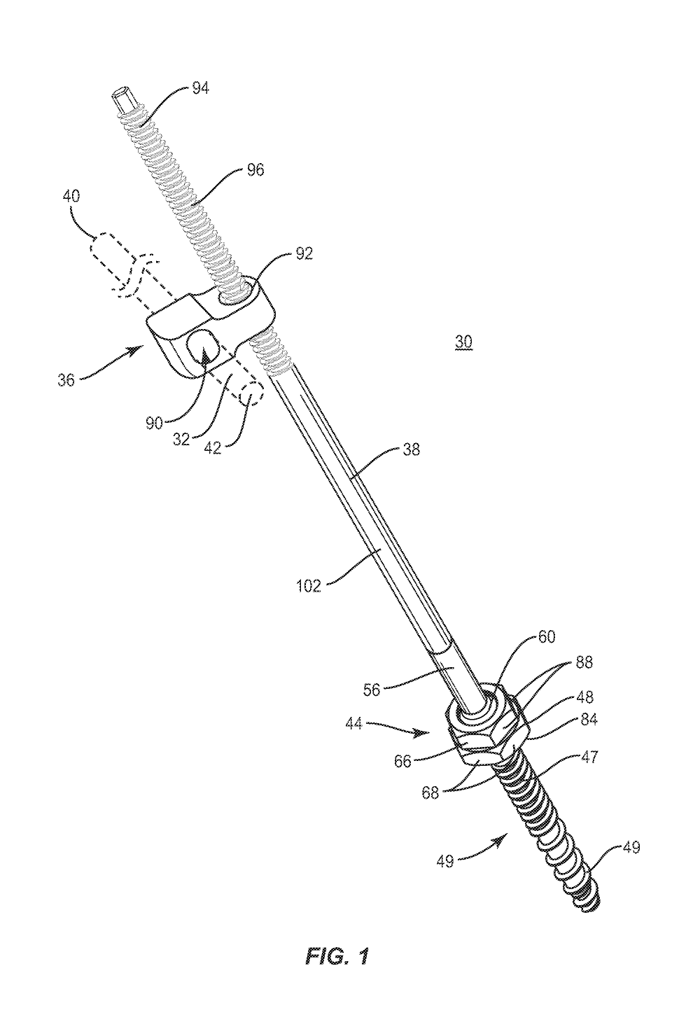

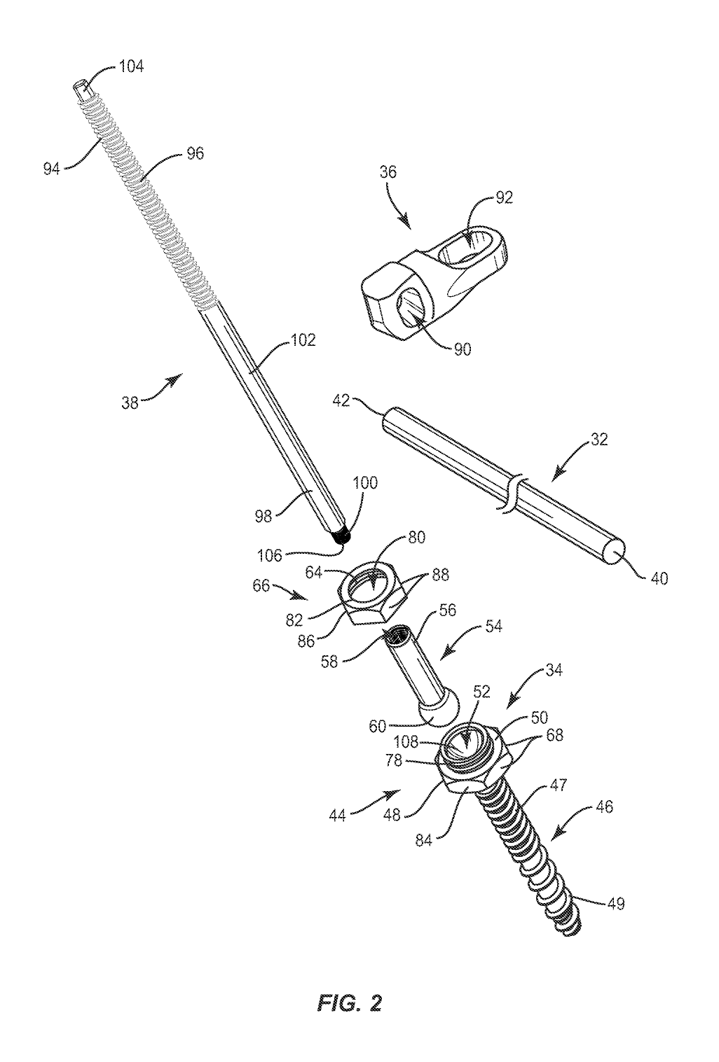

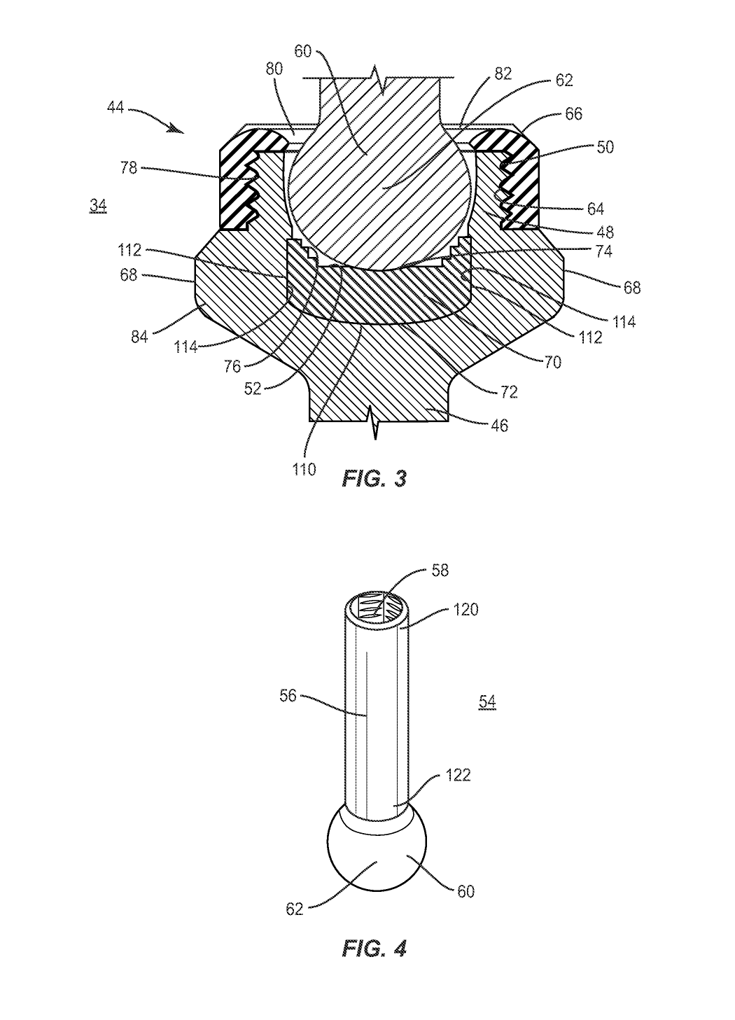

The following discussion includes a description of a spinal correction system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there is illustrated components of a system, such as, for example, a spinal correction system 30.

The components of system 30 can be fabricated from biologically acceptable materials suitable for medical applications, including metals, synthetic polymers, ceramics, bone material, tissue and/or their composites, depending on the particular application and/or preference of a medical practitioner. For example, the components of system 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 30 may have material composites, including the above materials, to achieve various desired characteristics such as strength, rigidity, elasticity, compliance, biomechanical performance, durability and radiolucency or imaging preference. The components of system 30, individually or collectively, may also be fabricated from a heterogeneous material such as a combination of two or more of the above-described materials. The components of system 30 may be monolithically formed, integrally connected or include fastening elements and/or instruments, as described herein.

System 30 is employed, for example, with an open, mini-open or minimally invasive surgical technique to attach a longitudinal element to a first side, such as, for example, a convex side of a spine that has a spinal disorder. In one embodiment, the longitudinal element may be affixed to the convex side of each of a plurality of vertebrae such that system 30 prevents growth of vertebrae of a selected section of the spine while allowing for growth and adjustments to a second side, such as, for example, a concave side of the plurality of vertebrae for a correction treatment to treat various spine pathologies.

System 30 includes at least one longitudinal element, such as, for example, a vertebral rod 32, at least one fixation element 34, a connector 36 and an elongated reduction element, such as, for example, a reduction post 38. Rod 32 is substantially cylindrical and extends between a first end 40 and a second end 42. In one embodiment, rod 32 is disposed to extend along an axial plane, such as for example, a sagittal plane of a body of a patient. It is contemplated that system 30 may include one or a plurality of longitudinal elements. It is further contemplated that the one or all of the plurality of longitudinal elements may be disposed in various relative orientations, such as, for example, side-by-side, parallel, transverse, perpendicular or angular and/or be disposed to extend along a coronal, sagittal and transverse planes of the body and geometric variations thereof.

Rod 32 has a uniform thickness/diameter. It is envisioned that rod 32 may have various surface configurations, such as, for example, rough, threaded for connection with surgical instruments, arcuate, undulating, porous, semi-porous, dimpled, polished and/or textured according to the requirements of a particular application. It is contemplated that the thickness defined by rod 32 may be uniformly increasing or decreasing, or have alternate diameter dimensions along its length. It is further contemplated that rod 32 may have various cross section configurations, such as, for example, oval, oblong, triangular, rectangular, square, polygonal, irregular, uniform, non-uniform, variable and/or tapered.

It is contemplated that rod 32 may have various lengths, according to the requirements of a particular application. It is further contemplated that rod 32 may be a tether, braided, such as a rope, or include a plurality of elongated elements to provide a predetermined force resistance. It is envisioned that rod 32 may be made from autograft and/or allograft and be configured for resorbable or degradable applications. In one embodiment, rod 32 is a cadaver tendon. In one embodiment, rod 32 is a tendon that may be harvested, for example, from a patient or donor. It is contemplated that a tendon harvested from a patient may be affixed in remote locations with the patient's body.

It is envisioned that all or only a portion of rod 32 may have a semi-rigid, flexible or elastic configuration and/or have elastic and/or flexible properties similar to the properties from materials, such as, for example, fabric, silicone, polyurethane, silicone-polyurethane, copolymers, rubbers, polyolefin rubber, elastomers, thermoplastic elastomers, thermoset elastomers and elastomeric composites. In one embodiment, rod 32 provides a selective amount of expansion and/or extension in an axial direction. It is contemplated that rod 32 may have a flexible configuration, which includes movement in a lateral or side to side direction. It is further contemplated that rod 32 may be compressible in an axial direction. Rod 32 can include a plurality of separately attachable or connectable portions or sections, such as bands or loops, or may be monolithically formed as a single continuous element.

Element 34 includes a proximal portion 44 and a distal portion 46 configured for penetrating tissue. Portion 46 has a cylindrical cross section configuration and includes an outer surface having engaging structures, such as, for example, an externally threaded surface. The externally threaded surface has a first thread form configuration 47 adapted for anchoring portion 46 in cortical bone and a second thread form configuration 49 adapted for anchoring portion 46 in cancellous bone. It is envisioned that the respective positions of configurations 47, 49 may be reversed.

Configuration 47 has a length that is approximately equivalent to a length of configuration 49. It is further envisioned that the respective lengths of configurations 47, 49 may vary depending upon the requirements of a particular application. It is contemplated that portion 46 may include one thread form configuration or a plurality of different thread form configurations. Configurations 47, 49 may be single pitch or multiple pitch. It is further contemplated that configuration 47 and/or configuration 49 may include a single thread turn or a plurality of discrete threads. Configurations 47, 49 are continuous. It is envisioned that configurations 47, 49 may be non-continuous such that there is gap between configurations 47, 49. It is further envisioned that other engaging structures may be located on portion 46, in place of or in addition to a the thread form discussed above, such as, for example, a nail configuration, barbs, expanding elements, raised elements and/or spikes to facilitate engagement of portion 46 with tissue, such as, for example, vertebrae. In one embodiment, portion 44 comprises cobalt chrome and portion 46 comprises titanium or a titanium alloy. It is contemplated that all or only a portion of portion 44 and/or portion 46 may comprise additional or alternative materials such as, for example, metals, synthetic polymers, ceramics, bone material, tissue and/or their composites, depending on the particular application and/or preference of a medical practitioner.

It is envisioned that all or only a part of portion 46 may have alternate cross section configurations, such as, for example, oval, oblong, triangular, square, polygonal, irregular, uniform, non-uniform, offset, staggered, undulating, arcuate, variable and/or tapered. It is contemplated that the outer surface of portion 46 may include one or a plurality of openings. It is further contemplated that all or only a portion of the outer surface of portion 46 may have alternate surface configurations to enhance fixation with tissue such as, for example, rough, arcuate, undulating, mesh, porous, semi-porous, dimpled and/or textured according to the requirements of a particular application. It is envisioned that all or only a part of portion 46 may be cannulated. It is further envisioned that portion 46 may include one or a plurality of hooks, anchors, tissue penetrating screws, mono-axial screws, multi-axial screws, expanding screws, wedges, buttons, clips, snaps, friction fittings, compressive fittings, expanding rivets, staples, nails, adhesives, fixation plates and/or posts. These fixation elements may be coated with an osteoinductive or osteoconductive material to enhance fixation, and/or include one or a plurality of therapeutic agents.

Portion 44 includes a wall, such as, for example, a collar 48 having an outer surface 50 and an inner surface 108 configured for disposal of a compression element 70. Inner surface 108 includes a concave bottom surface 110 extending between planar side surfaces 112 extending perpendicular to surface 110 such that inner surface 108 has a substantially U-shaped cross section. It is envisioned that all or only a portion of inner surface 108, including surfaces 110, 112, may be variously configured and dimensioned, such as, for example, planar, concave, convex, polygonal, irregular, uniform, non-uniform, staggered, tapered, consistent or variable, depending on the requirements of a particular application.

Portion 44 includes a post 54 having a substantially cylindrical first portion, such as, for example, a shaft 56 having an inner threaded surface 58. Shaft 56 is an elongate rigid member that extends between a first end 120 and a second end 122, and has an exterior surface, which is smooth and continuous. Post 54 is configured for engagement with connector 36 to link rod 32 to element 34, as will be described. It is envisioned that shaft 56 may be variously configured and dimensioned, such as, for example, round, oval, oblong, square, rectangular, polygonal, irregular, uniform, non-uniform, offset, staggered, tapered, consistent, variable, planar, concave or convex, depending on the requirements of a particular application. Surface 58 is configured to engage a portion of post 38 to retain post 38 with post 54, as will be discussed.

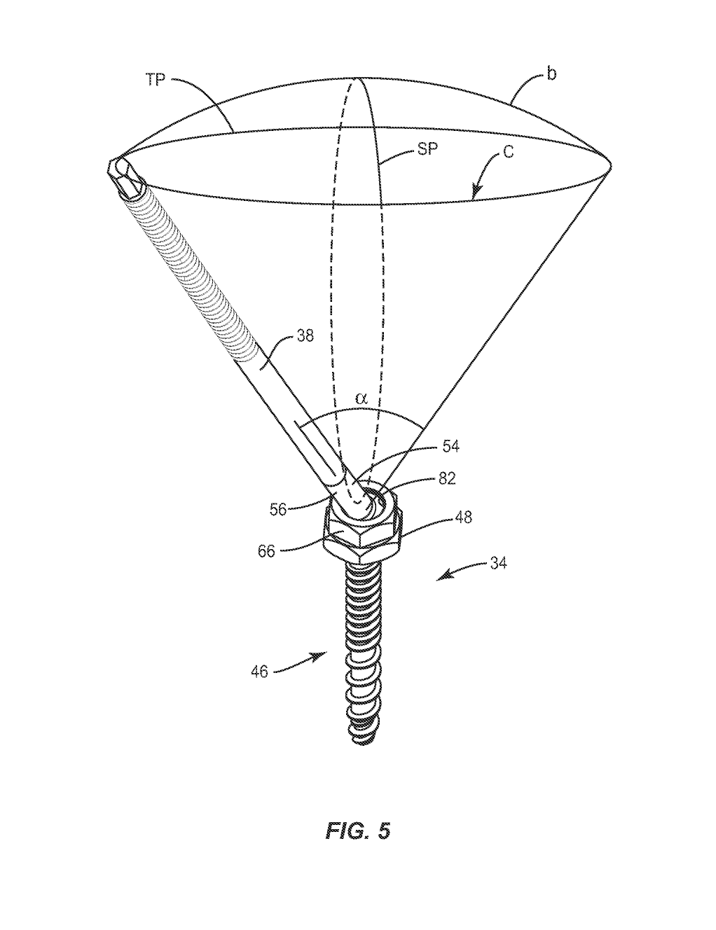

Portion 44 includes a substantially spherical second portion, such as, for example, a ball 60 that is connected with second end 122. Ball 60 is monolithically formed with shaft 56 and defines a center 62 such that first end 120 is rotatable to a selected angle through and within an angular range a about center 62 in a plurality of planes relative to distal portion 46, as shown in FIG. 5, and described below.

Compression element 70 is disposed between inner surface 108 and ball 60. An outer dimension of element 70 is smaller than an inner dimension of inner surface 108 such that element 70 is fixed in collar 48. Element 70 includes a convex first surface 72 that engages surface 110 such that no gap exists between surfaces 72, 110 when element 70 is disposed in collar 48. Element 70 includes planar side surfaces 114 configured to engage surfaces 112 when element 70 is disposed in collar 48 to securely retain element 70 such that movement of element 70 relative to collar 48 is prevented. It is contemplated that surface 72 may include a friction enhancing surface, such as, for example, roughened, knurled, porous, semi-porous, dimpled and/or textured for fixation with surface 110, according to the requirements of a particular application.

Element 70 includes a concave second surface 74 opposite surface 72 configured to engage ball 60. Surface 74 and inner surface 108 form a socket 52 configured for disposal of ball 60. Ball 60 slidably engages the surfaces of socket 52 and is movable relative to socket 52 in a ball and socket configuration such that first end 120 is rotatable to a selected angle through and within angular range a about center 62 in a plurality of planes relative to distal portion 46. The ball and socket joint facilitates free rotation of shaft 56 in a first orientation of element 34 through and within angular range a in the plurality of planes. It is envisioned that socket 52 and/or ball 60 may be variously configured and dimensioned, such as, for example, round, oval, oblong, square, rectangular, polygonal, irregular, uniform, non-uniform, offset, staggered, tapered, consistent, variable, concave, and/or convex. It is further envisioned that socket 52 and/or ball 60 may form an ellipsoid joint, a saddle joint, a hinge joint or a pivot joint, depending on the requirements of a particular application.

Surface 74 includes at least one ridge 76 configured to engage ball 60. In a second orientation of element 34, as described herein, ball 60 is driven into a locking engagement with surface 74 such that ridges 76 are pressed into surface 74 and shaft 56 is fixed in an orientation relative to portion 46. It is contemplated that element 70 may be fabricated from a flexible or elastic material and/or a softer material relative to ball 60 to enhance slidable engagement and/or fixation upon application of a compressive force thereto. This configuration allows ball 60 to depress ridges 76 when ball 60 engages element 70 thereby providing a more definite fixation between ball 60 and element 70.

A portion 78 of surface 50 is threaded and configured to engage a portion of a continuous threaded inner surface 64 of a locking element, such as, for example, a locking nut 66. Nut 66 is initially threaded with collar 48 to retain ball 60 in socket 52 in a provisional locking engagement and to form the ball and socket configuration of element 34. In the provisional locking engagement, the components of the ball and socket joint are not compressed and permitted to freely rotate. In the provisional locking engagement, element 34 is disposed in the first orientation and shaft 56 freely rotates through and within angular range a in the plurality of planes such that ball 60 slidably engages the surfaces of socket 52 and is movable relative to socket 52.

Rotation of locking nut 66 relative to collar 48 in a first direction, such as, for example, clockwise, from the provisional locking engagement advances locking nut 66 distally relative to element 34 such that locking nut 66 is tightened onto collar 48. Nut 66 is tightened with collar 48 to the second orientation such that shaft 56 is fixed in an orientation relative to portion 46. Locking nut 66 is rotatable relative to collar 48 in a second direction, opposite the first direction, such as, for example, counterclockwise, to advance locking nut 66 proximally relative to collar 48 such that locking nut 66 is loosened to the provisional locking engagement for further positioning and/or for removal from element 34. It is contemplated that locking nut 66 may be retained with collar 48 in alternate fixation configurations, such as, for example, friction fit, pressure fit, locking protrusion/recess, locking keyway and/or adhesive.

Locking nut 66 defines a circular opening 80 and a radial perimeter 82 that defines a range of motion of shaft 56 in the provisional locking engagement orientation. An outer surface 86 of locking nut 66 includes a plurality of planar faces 88 configured for engagement with a surgical tool to rotate nut 66 relative to collar 48, as described.

Surfaces 74, 108 and nut 66 form socket 52 for disposal of ball 60, and perimeter 82 of opening 80 defines the range of motion shaft 56 as facilitated by movement of ball 60 in the ball and socket configuration. In one embodiment, as shown in FIG. 5, nut 66 and collar 48 are disposed in the provisional locking engagement orientation. Shaft 56 is rotatable to a selected angle through and within angular range a about center 62 relative to distal portion 46 in a plurality of planes that lie in a cone configuration C. The area and/or volume defined by cone C, which includes the configuration disposed between center 62 and a circular base b, is defined by the range of motion of shaft 56 within perimeter 82 about center 62. The plurality of planes includes a locus of all straight line segments that join the center 62 to base b. For example, shaft 56 is separately rotatable to a selected angle within angular range a in each plane corresponding to a particular straight line segment that lies in cone configuration C. It is contemplated that shaft 56 may rotatable through the individual diameters, chords, section and/or radii of base b and/or other portions of cone C.

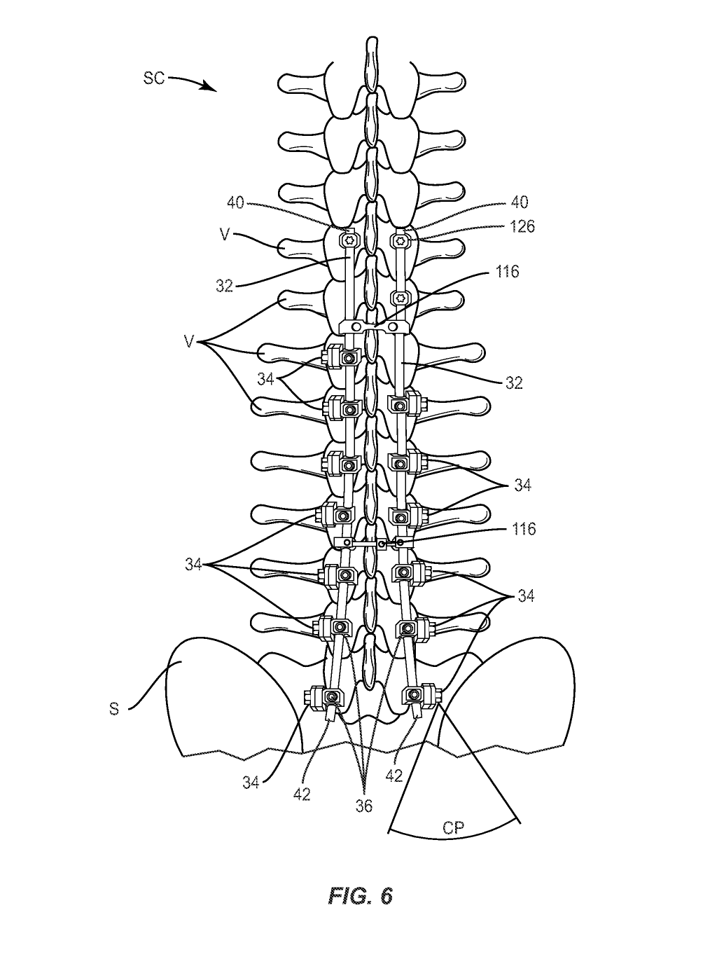

In one embodiment, shaft 56 is rotatable to a selected angle within angular range a in a sagittal plane SP of a body of a patient, corresponding to a particular plane that lies in cone C. In one embodiment, shaft 56 is rotatable to a selected angle within angular range a in a transverse plane TP of the body, corresponding to a particular plane that lies in cone C. In one embodiment, shaft 56 is rotatable to a selected angle within angular range a in a coronal plane CP (FIG. 6) of the body, corresponding to a particular plane that lies in cone C.

A lower portion 84 of surface 50 includes a plurality of planar faces 68 configured for engagement with a surgical tool to rotate collar 48 and drive and/or axially translate portion 46 into tissue, such as, for example, vertebrae. Connector 36 connects element 34 and rod 32 and includes a first cavity 90 configured for disposal of rod 32 and a second cavity 92 configured for disposal of shaft 56. Cavity 90 is disposed transverse to cavity 92. Cavity 90 has a substantially circular cross sectional configuration to accommodate rod 32. Cavity 92 has a substantially circular cross sectional configuration to accommodate shaft 56. It is envisioned that all or only a portion of cavity 90 and/or cavity 92 may have alternate cross section configurations, such as, for example, oval, oblong, triangular, square, polygonal, irregular, uniform, non-uniform, offset, staggered, undulating, arcuate, variable and/or tapered.

Post 38 is a rigid elongate member and extends between a first end 94 having a first thread form 96 and a second end 98 having a second thread form 100 of a size different than thread form 96. Post 38 includes an intermediate section 102 between ends 94, 98 having an exterior surface, which is smooth, continuous and free of threads. A diameter of the exterior surface of post 38 is less than a diameter of cavity 92 such that post 38 may be inserted therein to reduce connector 36 adjacent to element 34 at a surgical site for connection of connector 36 with element 34. Thread form 96 is configured to allow for the attachment of instruments, such as, for example, reduction instruments with post 38 and thread form 100 is configured to mate with the threads of surface 58 to engage posts 38, 54. While post 38 has been described as a separate member from post 54, posts 38 and 54 may be monolithically formed. A post cutter may then be used to remove any length of post after reduction is complete.

End 98 includes a cylindrical engagement portion 106 having a diameter that is less than a diameter of surface 58 such that portion 106 may be inserted into surface 58 and positioned such that thread form 100 is aligned with the threads of surface 58. Post 38 may be rotated relative to element 34 in the first direction such that thread form 100 engages the threads of surface 58 to engage posts 38, 54 such that posts 38, 54 are coaxial. Rotating post 38 relative to element 34 in the second direction permits removal of post 38 from surface 58. It is envisioned that post 38 may engage post 54 in alternate fixation configurations, such as, for example, friction fit, pressure fit, locking protrusion/recess, locking keyway and/or adhesive.

The diameter of an exterior surface of post 38 is approximately equivalent to the diameter of an exterior surface of shaft 56 such that the exterior surfaces of post 38 and shaft 56 are aligned when posts 38, 54 are engaged with one another such that connector 36 can be slid down post 38 and onto post 54, where it may be used to secure a longitudinal element, such as, for example, rod 32. A reduction instrument may engage thread form 96 to facilitate reducing connector 36 from post 38 to post 54.

End 94 includes a drive portion 104 that allows portion 106 to be rotated and tightened into shaft 56 to engage post 38 with post 54. Portion 104 has a faceted cross-sectional profile, such as, for example, hexagonal, and has a diameter which is less than that of post 38. Post 38 has an overall length that is greater than that of post 54. It is envisioned that at least one of the components of system 30, including, for example, rod 32, element 34, connector 36 and/or post 38 can be provided in a number of sizes and configurations, including varying lengths and diameters.

In assembly, operation and use, system 30 is employed with a surgical procedure, such as, for example, a correction treatment for a spinal disorder. It is contemplated that one or all of the components of system 30 can be delivered or implanted as a pre-assembled device or can be assembled in situ. System 30 may be completely or partially revised, removed or replaced. System 30, as shown in FIG. 6, can be employed with a surgical correction treatment of an applicable condition or injury of an affected section of a spinal column segment SC and adjacent areas within a body, such as, for example, vertebrae V and/or a sacrum S.

To treat segment SC, a medical practitioner obtains access to a surgical site including vertebrae V and sacrum S in any appropriate manner, such as through incision and retraction of tissues. It is envisioned that system 30 can be used in any existing surgical method or technique including open surgery, mini-open surgery, minimally invasive surgery and percutaneous surgical implantation, whereby vertebrae V and/or sacrum S is accessed through a mini-incision, or sleeve that provides a protected passageway to the area. Once access to the surgical site is obtained, the particular surgical procedure can be performed for treating the spine disorder. The configuration and dimension of rod 32 is determined according to the configuration and dimension of selected section of segment SC and the requirements of a particular application.

An incision is made in the body of a patient and a cutting instrument (not shown) creates a surgical pathway for implantation of components of system 30. A preparation instrument (not shown) can be employed to prepare tissue surfaces of vertebrae V and/or sacrum S, as well as for aspiration and irrigation of a surgical region according to the requirements of a particular surgical application.

Pilot holes or the like are made in vertebrae V and/or sacrum S for receiving fixation elements, including elements 34 and mono-axial screws 126. Elements 34 and mono-axial screws 126 are affixed with segment SC via torque and rotation of the threaded shaft into the tissue of vertebrae V and/or sacrum S, according to the requirements of a particular application.

For each element 34, nut 66 is initially threaded with collar 48 to retain ball 60 in socket 52 in a provisional locking engagement, as described. In the provisional locking engagement, element 34 is disposed in the first orientation and shaft 56 freely rotates through and within angular range a in the plurality of planes such that ball 60 slidably engages the surfaces of socket 52 and is movable relative to socket 52.

Rod 32 is inserted through cavities 90 of connectors 36 to be affixed with elements 34 along segment SC to form a portion of the surgical treatment construct. Post 38 is connected with post 54, as described. Shaft 56 and post 38 are rotated to a selected angle through and within angular range a about center 62 relative to distal portion 46 in a plurality of planes, as described. For example, shaft 56 and post 38 are rotated in one or a plurality of planes to align shaft 56 and post 38 with cavity 92 so that the surgical treatment construct including connector 36 and rod 32 can be reduced to adjacent the surgical site, segment SC and elements 34. Upon positioning shaft 56 and post 38 in a desired orientation to facilitate alignment, nut 66 is tightened with collar 48 to the second orientation, as described, such that shaft 56 and post 38 are fixed in the orientation relative to portion 46. Locking nut 66 is rotatable relative to collar 48 from the second orientation to the provisional locking engagement for further positioning and/or for removal from element 34.

End 94 is inserted through cavity 92 such that connector 36 may be advanced distally toward post 54. Connector 36 is advanced distally along post 38 until shaft 56 is inserted through cavity 92. Element 34 is affixed with connector 36 via a set screw. It is envisioned that a reduction instrument (not shown) may engage thread form 96 to facilitate reduction of connector 36 from post 38 onto post 54. Rod 32 is affixed with screws 126 via set screws.

It is envisioned that connector 36 may engage post 54 before or after shaft 56 is rotated to and/or fixed in the selected position and that rod 32 may be disposed in cavity 90 before or after shaft 56 is rotated to and/or fixed in the selected position. It is further envisioned that shaft 56 may be disposed in cavity 92 before or after shaft 56 is rotated to and/or fixed in the selected position. Rods 32 are positioned on opposite sides of the sagittal plane of the patient and extend in the cephalad-caudal direction to provide bi-lateral stabilization. Rods 32 are connected by one or more crosslink devices 116, which extend laterally within a coronal plane CP to provide additional stabilization for treating spinal disorders.

In one embodiment, system 30 includes a plurality of alternatively sized and/or configured posts, similar to post 54. It is envisioned that the posts may be of shorter lengths, intermediate lengths and greater lengths for adjusting the proximity of a spinal rod and/or connector with element 34 and/or vertebrae.

It is contemplated one or a plurality of fixation elements, such as, for example, element 34 may be employed with a single vertebral level. It is further contemplated that the fixation elements may be engaged with vertebrae in various orientations, such as, for example, series, parallel, offset, staggered and/or alternate vertebral levels. It is envisioned that the fixation elements may include one or a plurality of anchors, tissue penetrating screws, conventional screws, expanding screws, wedges, anchors, buttons, clips, snaps, friction fittings, compressive fittings, expanding rivets, staples, nails, adhesives, posts, fixation plates and/or posts. These fixation elements may be coated with an osteoinductive or osteoconductive material to enhance fixation, and/or include one or a plurality of therapeutic agents.

In one embodiment, spinal correction system 30 includes an agent, which may be disposed, packed or layered within, on or about the components and/or surfaces of spinal correction system 30. It is envisioned that the agent may include bone growth promoting material, such as, for example, bone graft to enhance fixation of the fixation elements with vertebrae V. It is contemplated that the agent may include one or a plurality of therapeutic agents and/or pharmacological agents for release, including sustained release, to treat, for example, pain, inflammation and degeneration. The components of spinal correction system 30 can be made of radiolucent materials such as polymers. Radiomarkers may be included for identification under x-ray, fluoroscopy, CT or other imaging techniques. It is envisioned that the use of microsurgical and image guided technologies may be employed to access, view and repair spinal deterioration or damage, with the aid of spinal correction system 30. Upon completion of the procedure, the surgical instruments and assemblies are removed and the incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal correction system comprising:
at least one longitudinal element;
at least one fixation element including a proximal portion and a distal portion configured for penetrating tissue, the proximal portion including a wall that defines a cavity and at least one post including a first portion and a second portion having a center,
the at least one fixation element further including a locking element engageable with the wall to dispose the second portion in the cavity; and
a connector defining a first cavity configured for disposal of the at least one longitudinal element and a second cavity configured for disposal of the first portion,
wherein the at least one fixation element is moveable between a first orientation such that the first portion is rotatable to a selected angle about the center in a plurality of planes relative to the distal portion and a second orientation such that the locking element fixes the first portion in a selected position relative to the distal portion.

2. A spinal correction system as recited in claim 1, wherein the second portion is disposed in the cavity such that the first portion is freely rotatable in a conical configuration.

3. A spinal correction system as recited in claim 1, wherein the plurality of planes include a coronal plane of a body, a transverse plane of the body and a sagittal plane of the body.

4. A spinal correction system as recited in claim 1, wherein the second portion is substantially spherical.

5. A spinal correction system as recited in claim 1, wherein the wall includes a concave surface that defines the cavity.

6. A spinal correction system as recited in claim 1, wherein the second portion and the cavity form a ball and socket joint.

7. A spinal correction system as recited in claim 1, wherein the locking element includes an inner surface that defines an opening, the inner surface defining a perimeter of the opening, the perimeter defining a range of motion of the first portion including the selected angle about the center in the plurality of planes.

8. A spinal correction system as recited in claim 1, wherein the locking element includes a continuous inner surface that defines a circular opening, the inner surface defining a radial perimeter of the opening, the perimeter defining a range of motion of the first portion including the selected angle about the center in the plurality of planes.

9. A spinal correction system as recited in claim 1, further comprising a compression element disposed between the wall and the second portion.

10. A spinal correction system as recited in claim 1, further comprising a compression element including a first surface configured to engage the wall and a second concave surface configured to engage the second portion.

11. A spinal correction system as recited in claim 1, further comprising a compression element including a first surface having a stepped configuration for engaging the wall and a second concave surface configured to engage the second portion.

12. A spinal correction system as recited in claim 1, further comprising a compression element disposed between the wall and the second portion, the compression element including a first surface and a second surface having at least one ridge.

13. A spinal correction system as recited in claim 1, wherein the wall includes a threaded outer surface and the locking element includes a threaded inner surface configured to engage the threaded outer surface.

14. A spinal correction system as recited in claim 1 wherein the first portion includes an inner threaded surface and further comprising a reduction element having a threaded portion engageable with the inner threaded surface.

15. A spinal correction as recited in claim 1, wherein the distal portion comprises titanium, a titanium alloy, or a combination thereof.

16. A spinal correction system as recited in claim 1, further comprising a plurality of alternately sized and/or configured posts.

17. A spinal correction system as recited in claim 1, further comprising an elongated reduction element having a thread form, the first portion including a threaded inner surface configured to mate with the thread form.

18. A method for treating a spinal disorder, the method comprising the steps of:
providing a spinal correction system comprising:
a longitudinal element,
at least one fixation element including a proximal portion and a distal portion, the proximal portion including a wall that defines a cavity and at least one post including a first portion and a second portion having a center,
the at least one fixation element further including a locking element engageable with the wall to retain the second portion in the cavity, and
a connector defining a first cavity and a second cavity;
attaching the distal portion with tissue;
provisionally engaging the locking element with the wall;
disposing the longitudinal element in the first cavity;
rotating the first portion in a first orientation to a selected angle about the center in a plurality of planes relative to the distal portion to a selected position such that the first portion is aligned with the second cavity;
engaging the locking element with the wall to fix the first portion in a second orientation in the selected position; and
disposing the first portion in the second cavity.

19. A method as recited in claim 18, wherein in the first orientation the second portion is disposed in the cavity such that the first portion is freely rotatable in a conical configuration.

20. A spinal correction system comprising:
at least one longitudinal element;
at least one fixation element including a proximal portion and a distal portion configured for penetrating tissue, the proximal portion including a collar having a threaded outer surface and defining a concave socket, the proximal portion further including a post including a shaft having an inner threaded surface and being integrally connected with a ball having a center,
the at least one fixation element further including a locking nut having an inner threaded surface that defines a circular opening having a radial perimeter defining a range of motion of the first portion, the threaded inner surface of the locking nut being configured to mate with the threaded outer surface of the collar to dispose the ball in the socket,
the at least one fixation element further including a compression element disposed between the collar and the ball, the compression member including a first surface engageable with the collar and a second concave surface engageable with the ball, the second surface including a stepped surface;
a connector defining a first cavity configured for disposal of the at least one longitudinal element and a second cavity configured for disposal of the first portion, the first cavity being disposed in a transverse orientation relative to the second cavity; and
an elongated reduction element having a threaded portion engageable with the inner threaded surface of the first portion,
wherein the fixation element is moveable between a first orientation such that the shaft is rotatable through a selected angle about the center in a plurality of planes relative to the distal portion and a second orientation such that the locking element fixes the shaft in a selected position relative to the distal portion.
